(19) United States
(12) Patent Application Publication
(10) Pub. No.: US 2007/0078339 A1
(43) Pub. Date: Apr. 5, 2007

(54) Control of hybrid SPECT study definition via patient positioning monitors

(76) Inventor: Keith Andress, Barrinton, IL (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/450,205

(22) Filed: Jun. 9, 2006

Related U.S. Application Data
(60) Provisional application No. 60/722,202, filed on Sep. 30, 2005.

(51) Int. Cl. A61B 5/05; A61B 6/00

(52) U.S. Cl. 600/436; 600/407

(57) ABSTRACT

A method of specifying the anatomy to be covered by a hybrid scan (e.g., a SPECT/CT scan) is presented in which a monitor of a first imaging device (e.g., a SPECT Patient Positioning Monitor) is used to control both portions of the scan (e.g., both a SPECT and a CT portion of the scan).

### PRIORITY CLAIM TO RELATED APPLICATION

This application claims priority from U.S. Provisional Patent Application Ser. No. 60/722,202, filed Sep. 30, 2005, the disclosure of which is incorporated by reference in its entirety herein.

### BACKGROUND

1. Field of the Invention

The present invention relates in general to medical imaging systems. More particularly, the present invention relates to detectors for gamma cameras of nuclear medicine imaging systems and the like.

2. Background Discussion

A variety of medical imaging systems are known. Some illustrative imaging systems include nuclear medical imaging systems (e.g., gamma cameras), computed tomography (CT or CAT) systems, magnetic resonance imaging (MRI) systems, positron-emission tomography (PET) systems, ultrasound systems and/or the like.

With respect to nuclear medical imaging systems, nuclear medicine is a unique medical specialty wherein radiation (e.g., gamma radiation) is used to acquire images that show, e.g., the function and/or anatomy of organs, bones and/or tissues of the body. Typically, radioactive compounds, called radiopharmaceuticals or tracers, are introduced into the body, either by injection or ingestion, and are attracted to specific organs, bones or tissues of interest. These radiopharmaceuticals produce gamma photon emissions that emanate from the body and are captured by a scintillation crystal, with which the photons interact to produce flashes of light or “events.” These events can be detected by, e.g., an array of photo-detectors, such as photomultiplier tubes, and their spatial locations or positions can be calculated and stored. In this manner, an image of an organ, tissue or the like under study can be created from the detection of the distribution of the radioisotopes in the body. Typically, one or more detectors are used to detect the emitted gamma photons, and the information collected from the detector(s) is processed to calculate the position of origin of the emitted photon from the source (i.e., the body organ or tissue under study). The accumulation of a large number of emitted gamma positions allows an image of the organ or tissue under study to be displayed.

FIG. 1(A) depicts components of a typical nuclear medical imaging system 100 (i.e., having a gamma camera or a scintillation camera) which includes a gantry 102 supporting one or more detectors 108 enclosed within a metal housing and movably supported proximate a patient 106 located on a patient support (e.g., pallet) 104. Typically, the positions of the detectors 108 can be changed to a variety of orientations to obtain images of a patient's body from various directions. In many instances, a data acquisition console 200 (e.g., with a user interface and/or display) is located proximate a patient during use for a technologist 107 to manipulate during data acquisition. In addition to the data acquisition console 200, images are often developed via a processing computer system which is operated at another image processing computer console including, e.g., an operator interface and a display, which may often be located in another room, to develop images. By way of example, the image acquisition data may, in some instances, be transmitted to the processing computer system after acquisition using the acquisition console.

Nuclear medicine imaging typically involves the assessment of a radionuclide distribution within a patient after the in vivo administration of radiopharmaceuticals. Imaging systems that assess radionuclide distribution include radiation detectors and acquisition electronics. Typically, the imaging systems detect x-ray or gamma ray photons derived from the administered radionuclides. Single photon emission imaging and coincidence imaging are two forms of nuclear medicine imaging that are currently in common use. In single photon emission imaging, the radionuclide itself directly emits the radiation to be assessed. For example, in Single Photon Emission Computed Tomography (SPECT), v-emitting radionuclides such as ^{99m}Tc, ¹²³I, ⁶⁷Ga and ¹¹¹In may be part of the administered radiopharmaceutical.

Detectors used in such single photon emission imaging often use collimators placed between the patient and the gamma ray camera of the detector. In general, the collimators help to eliminate substantially all photons but those photons traveling in a desired direction. For example, a parallel hole collimator helps to eliminate photons traveling in all directions except those almost perpendicular to the surface of the detector. The energy of emitted photons as well as their location of origin may then be accumulated until a satisfactory image is obtained.

Coincidence imaging helps to eliminate the need for such a collimator by relying on the detection of two photons at different detectors at nearly the same time. An example of coincidence imaging in current clinical use is Positron Emission Tomography (PET).

Typically, radiation detectors used in nuclear medicine imaging need to absorb x- or gamma-ray photons in an energy range typically between 1 keV and several MeV. These imaging photons are the photons either directly emitted or resulting from radionuclides within a patient. In order to stop imaging photons of these energies with a collimator in SPECT imaging, a material with a high density and a high atomic number (Z) is necessary. Lead is the most common material used for collimators, but other materials such as, e.g., tungsten may also be used.

Typically, in radiology, detectors used clinically only integrate the energy deposited by a beam. However, a new generation of detectors for digital radiography and computed tomography (CT) can obtain extra information by counting individual photons and measuring their energy.

With respect to scintillators, a variety of scintillators are known. For example, scintillators include, e.g., continuous single slab, pixilated and/or columnar grow crystals. As for radionuclide imagers with pixilated radiation detector elements, typically cadmium zinc telluride (“CZT”) crystals have recently been developed. In these pixilated radionuclide imagers, the intrinsic spatial resolution is defined by the size of the individual pixilated detector elements, rather than the separation between collimator holes. See, e.g., U.S. Pat. No. 6,838,672, assigned to the present assignee, the entire disclosure of which is incorporated herein by reference. With respect to the use of CZT as a solid state (i.e., semiconductor) detector material, as a single photon detector, CZT is typically superior to Nal in several performance parameters. Among other things, the count rate capability for CZT detectors is virtually unlimited as compared to a typical scintillator crystal, because each pixel (or picture element) of the CZT material can act as an independent detector. Thus, unlike a typical scintillator crystal, in which two events occurring very close in time and spatial location will produce overlapping light output, two gamma photons arriving at exactly the same time in adjacent pixels of a CZT detector could be independently detected and measured accurately with respect to energy, given an optimum electronic circuitry design.

Patient positioning is an important step in the acquisition of medical diagnostic scans. It is typically important to cover all of the appropriate anatomy to allow complete diagnosis. However, including non-affected anatomy is not desired for a number of reasons: acquiring and reviewing these portions a) can waste time, b) can decrease patient throughput, and, notably, c) for scans using ionizing radiation (such as, e.g., x-ray CT) can add unnecessary doses.

In the context of a hybrid diagnostic imager (e.g., having a combined SPECT and CT system), an even more significant problem exists in relation to correctly positioning a patient because the appropriate anatomy often is not completely apparent in either modality of the hybrid diagnostic imager independently.

Most currently available hybrid systems involve functional imaging devices (such as, e.g., PET and/or SPECT devices) combined with a CT device. These existing hybrid devices use the positioning aids provided by the CT device. That is, such existing hybrid devices use external markers (e.g., lasers and landmarks on the system) and the Topogram (Scout) image. After the Topogram has been acquired, the functional study is acquired to cover the same area. This existing method of positioning patients has a number of deficiencies:
1. This existing method does not use the functional information available from the combined device.
2. This existing method forces the acquisition of the CT scan to occur prior to the PET or SPECT scan.
3.For hybrid SPECT studies, the size of the SPECT FOV is fixed, while the CT scan length is not constrained. If, however, the CT scan is longer than the SPECT FOV, it is then impossible to cover the same area as the CT scan using a single SPECT FOV. In this case, the user must either select a sub-portion of the CT scan or plan additional SPECT FOVs to cover the full CT scan.

While a variety of background technologies exist, there is a continued need in the art for improved systems and methods.

### SUMMARY

The exemplary embodiments of the present invention can significantly improve upon existing methods and/or apparatuses.

According to some embodiments, a hybrid medical imaging system includes a first nuclear medical imaging device; a second imaging device configured to provide anatomical information of a patient independently from the nuclear medical imaging device; and a display configured to concurrently present images from both the first nuclear medical imaging device and the second imaging device to aid in patient positioning with respect to both the nuclear medical imaging device and the second imaging device.

In some examples, the system is configured such that images from the nuclear medical imaging device and from the second imaging device are displayed concurrently in an overlaying relationship on the display.

In additional examples, the second imaging device is configured to provide cross-sectional anatomical information of a patient.

In further examples, the second imaging device comprises a computed tomography (CT) device or a magnetic resonance imaging (MRI) device.

According to some other embodiments, a method of patient positioning in a hybrid medical imaging system having a) a nuclear medical imaging device and b) a second imaging device configured to provide anatomical information of a patient independently from the nuclear medical imaging device is performed that includes on a display concurrently presenting images from both the first nuclear medical imaging device and the second imaging device to aid in patient positioning with respect to both the nuclear medical imaging device and the second imaging device.

In some examples, the method further includes concurrently displaying images from the nuclear medical imaging device and the second imaging device in an overlaying relationship on the display.

In additional examples, the second imaging device is configured to provide cross-sectional anatomical information of a patient.

In further examples, the method further includes the steps of a) positioning a Field of View of the nuclear medical imaging device; and b) adapting a scan of the second medical imaging device with respect to the Field of View of the nuclear medical imaging device.

In still further examples, the step of adapting the scan of the second medical imaging device includes adapting the scan of the second medical imaging device to match the Field of View of the nuclear medical imaging device.

In some examples, the step of adapting the scan of the second medical imaging device includes adapting the scan of the second medical imaging device to a sub-portion of the Field of View of the nuclear medical imaging device.

In additional examples, the method further includes using line marker lines on the display to specify a sub-portion of a SPECT PPM image in which to acquire CT data.

In further examples, the method further includes limiting the extent of the scan of the second imaging device based on the sub-portion so as to limit dose delivered to the patient by the second imaging device.

In still further examples, the method further includes acquiring a Topogram with the second imaging device and using the Topogram in conjunction with SPECT PPM information for patient positioning with respect to the second imaging device.

In still further examples, using the Topogram in conjunction with SPECT PPM information includes displaying PPM information along with the Topogram image. In some examples, the method further includes displaying an extent of the PPM Field of View as an annotation overlaid on the Topogram.

In still further examples, the method further includes storing a PPM image and overlaying the PPM image with the Topogram.

In still further examples, the method further includes enabling the user to set the extent of a scan of the second imaging device.

In still further examples, the method further includes the system treating a Field of View determined by SPECT PPM images as initial settings for an extent of a scan of the second imaging device.

In still further examples, the method further includes the system enabling a user to modify the extent to refine the area to perform the scan.

In still further examples, the method further includes displaying an original PPM Field of View even when the user has modified the extent to enable a user to keep track thereof.

According to some other embodiments, a method for specifying the anatomy to be covered by a hybrid scan of a nuclear medical imaging device and a second imaging device includes controlling both portions of the scan with a Patient Positioning Monitor of the nuclear medical imaging device.

In some examples, the method further includes specifying the anatomy to be covered by a hybrid SPECT/CT scan including controlling both the SPECT and CT portions of the scan with a SPECT Patient Positioning Monitor.

The above and/or other embodiments, aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### BRIEF DESCRIPTION OF THE DRAWINGS

The exemplary embodiments of the present invention, as well as further objects, features and advantages of the embodiments will be more fully understood with reference to the following detailed description of the embodiments, when taken in conjunction with the accompanying drawings, wherein:

FIG. 1(A) is a schematic diagram of an illustrative and non-limiting nuclear medical imaging system in which some embodiments of the invention may be employed;

FIG. 1(B) is a schematic diagram of an illustrative hybrid SPECT and CT system within which aspects of the present invention can be incorporated in some illustrative and non-limiting embodiments;

FIG. 2 is a front view of an illustrative graphical user interface display that can be presented on a monitor according to exemplary embodiments of the invention; and

FIG. 3 is an architectural diagram depicting illustrative components of a computer that can be used to perform exemplary embodiments of the invention.

### DETAILED DESCRIPTION OF EXEMPLARY EMBODIMENTS

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein.

According to exemplary embodiments of the invention, in a hybrid imaging system novel features are employed that facilitate the definition of areas of interest—e.g., areas related to organs or the like that are clinically relevant to a particular set of circumstances. In exemplary embodiments, information from one system (e.g., a Nuclear Medical imaging system) is used to define areas of interest in a manner to avoid the need for an initial Topogram (such as, e.g., an initial computed tomography (CT) Topogram) for another system (e.g., a CT system). For reference, historically before the definition of a region for which a volume dataset was to be registered, for example by means of a spiral scan, an x-ray shadowgram (topogram) of the life form was usually produced. The definition of the region for which the volume dataset was to be registered ensued by means of a graphic marking of a region, that is generally a rectangular region in the topogram, which includes the region of the life form to be registered. See U.S. Pat. No. 6,865,249, assigned to Siemens Aktiengesellschaft, and U.S. Pat. No. 6,928,137, assigned to Siemens Aktiengesellschaft, the entire disclosures of which are incorporated herein by reference.

In some exemplary embodiments, hybrid devices formed by the combination of SPECT with a device providing anatomical information (such as, e.g., CT or MR) can be adapted so as to use a SPECT Patient Positioning Monitor (PPM or P-scope) to aid patient positioning. In this regard, a PPM can include, by way of example, any appropriate monitor, display or the like, and can include, e.g., a flat panel liquid crystal display (LCD) patient positioning computer monitor that is used to facilitate patient positioning (and, e.g., to provide start/stop commands at the patient side).

In some embodiments, aspects described herein are employed within hybrid imaging systems, such as, e.g., SIEMENS' TRUEPOINT SPECT CT hybrid imaging system that, among other things, combines the functional sensitivity of a SPECT with the detail of a multi-slice CT. Such hybrid devices have the potential to revolutionize diagnosis and treatment for various ailments, including, e.g., cancer, cardiac and neurological diseases. With a single scan, this imaging technology can be used to quickly capture comprehensive, accurate diagnostic information both on the molecular and anatomical levels. The SIEMENS TRUEPOINT SPECT CT technology enables clinicians to utilize the device in three ways to perform three separate studies: a) SPECT; b) multi-slice CT; and c) SPECT CT—all with a single system. In addition, the technology includes a flexible, scalable system architecture that allows for a variety of models, ranging from systems that offer attenuation correction to premium, multi-slice CT capabilities.

In such cases, it is assumed that the system geometry is known, such that the offset between the SPECT and CT Field of Views (FOVs) is known, in order to enable the registering of the SPECT data with the other modality. This geometric information is also used for various clinical applications. Accordingly, this requirement is not a burden for the implementation of the exemplary embodiments of the invention.

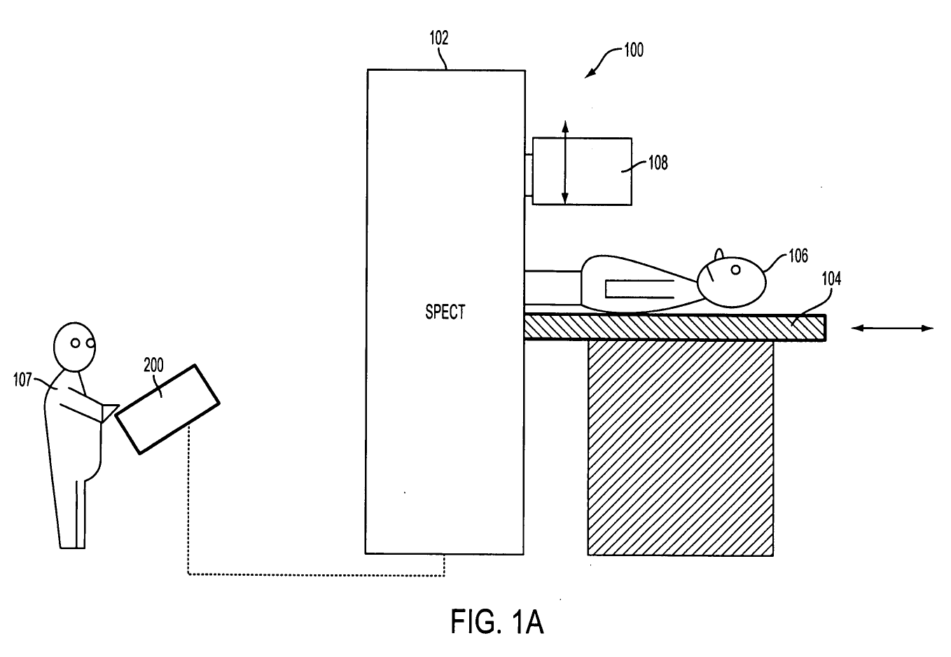

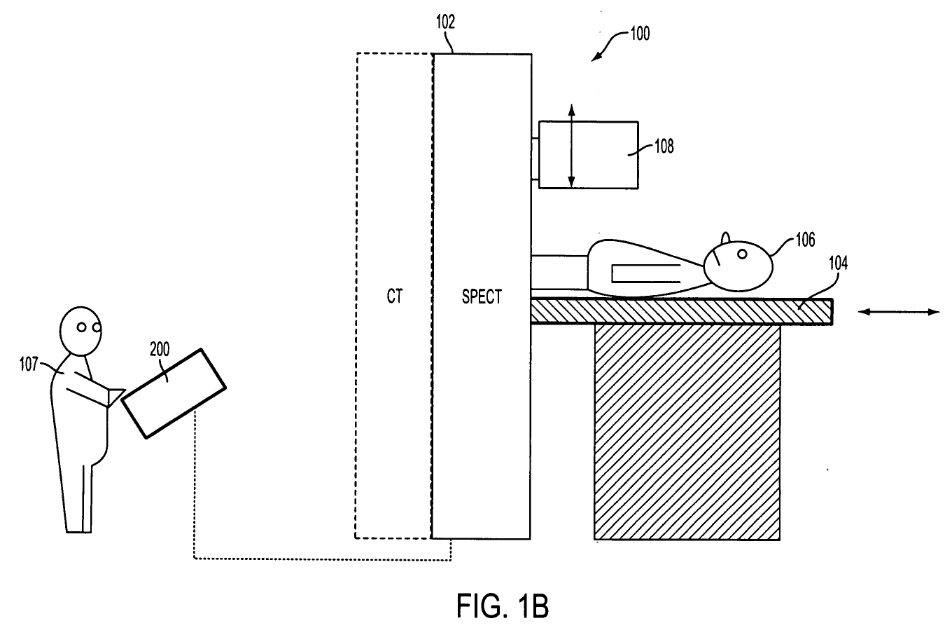

With reference to FIG. 1(B), in some embodiments, a hybrid system can include components similar to that shown in FIG. 1(A), along with elements related to the CT imaging components, such as, e.g., the CT imaging section shown in dashed lines in FIG. 1(B).

According to exemplary embodiments, the SPECT PPM can be adapted in a variety of ways to position a patient in a hybrid system either alone or in combination with, e.g., a CT Topogram.

In a first method, the FOV of the SPECT system can be positioned using the PPM in a manner similar to that which is currently done using stand-alone SPECT systems. Then, the extent of the CT scan can be matched to cover the FOV of the SPECT scan.

In a second method, the extent of the CT scan may be specified to be a sub-portion of that covered in the PPM. In various embodiments, any desired method of specifying can be employed, such as, by way of example, using marker lines on the PPM. In this manner, lines or the like can be used to specify a sub-portion of the PPM image in which to acquire CT data. Among other things, this can help to limit the amount of x-ray dose delivered to the patient by limiting the extent of the scan (e.g., in the axial direction).

In some embodiments of the above two cases, the only positioning information provided by the CT is the use of laser markers or physical landmarks. In some examples, if this information is not sufficient, then the system can be configured to allow the operator to acquire a CT Topogram to use in conjunction with the PPM information. In some embodiments, this can be performed routinely, or, alternatively, it can be included optionally, as needed. Accordingly, a third positioning method is contemplated as described below.

In the third method, the PPM can be used in conjunction with a Topogram (e.g., a CT Topogram) by displaying PPM information along with the Topogram image. In some examples, a simplified method of doing this can be to display the extent of the PPM FOV as an annotation overlaid on the Topogram image. For example, in some embodiments, the system is configured to enable a user to simply draw a box (such as, e.g., using any computer software Graphical User Interface (GUI) methodologies similar to that of other software applications) on the Topogram indicating the position of the SPECT FOV. In some more sophisticated methods, the PPM image is stored and that image is overlayed (e.g., using alpha blending or other techniques) with the Topogram. In some embodiments, the user can then set the extent of the CT scan using the registered data from both modalities.

In some embodiments, the CT FOVs determined by the SPECT PPM images are treated as initial settings for the extent of the CT scan. In exemplary embodiments, however, the system is configured to allow the user to modify these extents to further refine the area to perform the CT scan. In these cases, it is useful to display the original PPM FOV even when the user has modified the scan extent (e.g., this can be helpful to enable the user to keep track and avoid losing this “landmark” information).

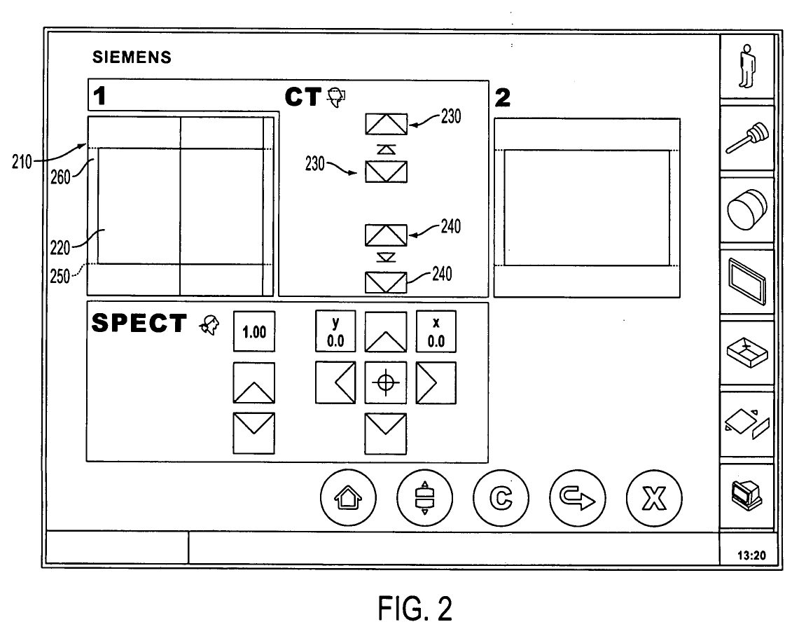

With reference to FIG. 2, an illustrative graphical user interface (GUI) is depicted which demonstrates aspects of the invention according to some exemplary embodiments. In the figure, the black areas 210 represent the PPM images. The displayed rectangle 220 shows the extent of the FOV on the Nuclear Medical (NM) detector.

When used for PPM planning, in exemplary embodiments, there are two more lines displayed on the image. Preferably, by default, one line 260 is aligned with the top of the rectangle 220 and comprises an upper line, and the other line 250 is aligned with the bottom of the rectangle 220 and comprises a bottom line. The CT scan covers the area between the lines 250 and 260. Thus, by default, the extent of the CT scan will be the full FOV of the NM detector.

As depicted in FIG. 2, a graphical user interface (GUI) is provided in which one can modify the extent of the CT scan. In this regard, in the illustrated embodiment, there are two sets of arrows 230 and 240 between the images. The upper set of arrows 230 can be used to position the upper line 260. The lower set of arrows 240 can be used to position the lower line 250. These arrows can be used to narrow the scan field to the area surrounding the organ of interest in order to limit dose.

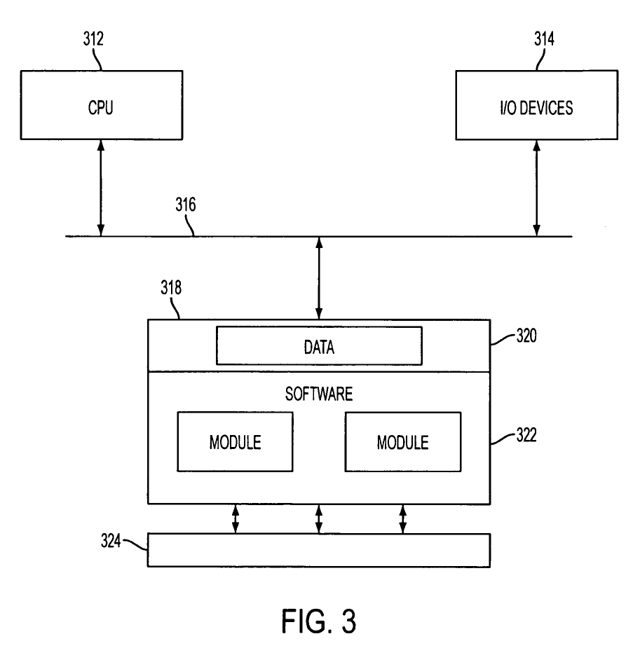

FIG. 3 shows components of an illustrative computer that can be used to implement computerized process steps in some embodiments of the invention. In some embodiments, the computer includes a central processing unit (CPU) 312, which can communicate with a set of input/output (I/O) device(s) 314 over a bus 316. The I/O devices 314 can include, for example, a keyboard, a mouse, a video monitor, a printer, and/or other devices. In some embodiments, the CPU 312 can communicate with a computer readable medium (e.g., conventional volatile or non-volatile data storage devices) 318 (hereafter “memory 318”) over the bus 316. The interaction between a CPU 322, I/O devices 324, a bus 316, and a memory 318 can be like that known in the art. Memory 318 can include, e.g., data 320 and software 322. The software 338 can include a number of modules 324 (two modules are depicted for illustrative purposes only) for implementing the steps of processes. Conventional programming techniques may be used to implement these modules.

In some embodiments, the various methods described herein may be implemented via one or more computer program product for use with a computer system. This implementation may, for example, include a series of computer instructions fixed on a computer readable medium (e.g., a diskette, a CD-ROM, ROM or the like) or transmittable to a computer system via and interface device, such as a modem or the like. The medium may be substantially tangible (e.g., communication lines) and/or substantially intangible (e.g., wireless media using microwave, light, infrared, etc.). The computer instructions can be written in various programming languages and/or can be stored in memory device(s), such as semiconductor devices (e.g., chips or circuits), magnetic devices, optical devices and/or other memory devices. In the various embodiments, the transmission may use any appropriate communications technology.

The preferred embodiments of the invention have wide applicability to various systems and devices, including, e.g., to hybrid imagers using SPECT, etc., to hybrid cameras for small animal imaging, and/or to various other hybrid devices, etc.

While illustrative embodiments of the invention have been described herein, the present invention is not limited to the various preferred embodiments described herein, but includes any and all embodiments having equivalent elements, modifications, omissions, combinations (for example, various aspects in different embodiments can be combined together when appropriate in various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” Means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited.

What is claimed is:

1. A hybrid medical imaging system, comprising:
a) a first nuclear medical imaging device;
b) a second imaging device configured to provide anatomical information of a patient independently from said nuclear medical imaging device; and
c) a display configured to concurrently present images from both said first nuclear medical imaging device and said second imaging device to aid in desired patient positioning with respect to both said nuclear medical imaging device and said second imaging device,
wherein a scan length of the second nuclear medical imaging device is adjusted to substantially match a coverage of a field of view of the first nuclear medical imaging device.

2. The system of claim 1, wherein said system is configured such that images from said nuclear medical imaging device and from said second imaging device are displayed concurrently in an overlaying relationship on said display.

3. The system of claim 1, wherein said second imaging device is configured to provide cross-sectional anatomical information of a patient.

4. The system of claim 2, wherein said second imaging device comprises a computed tomography (CT) device.

5. The system of claim 2, wherein said second imaging device comprises at least one of a magnetic resonance imaging (MRI) device, a positron emission tomography (PET), and a single photon emission computed tomography (SPECT).

6. A method of patient positioning in a hybrid medical imaging system having a) a nuclear medical imaging device and b) a second imaging device configured to provide anatomical information of a patient independently from said nuclear medical imaging device, comprising:
concurrently presenting on a display images from both said first nuclear medical imaging device and said second imaging device to aid in patient positioning with respect to both said nuclear medical imaging device and said second imaging device.

7. The method of claim 6, further including concurrently displaying images from said nuclear medical imaging device and said second imaging device in an overlaying relationship on said display.

8. The method of claim 6, wherein said second imaging device is configured to provide cross-sectional anatomical information of a patient.

9. The method of claim 6, wherein said second imaging device comprises a computed tomography (CT) device.

10. The method of claim 6, wherein said second imaging device comprises at least one of a magnetic resonance imaging (MRI) device, a positron emission tomography (PET), and a single photon emission computed tomography (SPECT).

11. The method of claim 6, further including the steps of:
a) positioning a Field of View of the nuclear medical imaging device; and
b) adapting a scan of the second medical imaging device with respect to the Field of View of the nuclear medical imaging device.

12. The method of claim 11, wherein said step of adapting the scan of the second medical imaging device includes adapting the scan of the second medical imaging device to match the Field of View of the nuclear medical imaging device.

13. The method of claim 11, wherein said step of adapting the scan of the second medical imaging device includes adapting the scan of the second medical imaging device to a sub-portion of the Field of View of the nuclear medical imaging device.

14. The method of claim 13, further including using line marker lines on the display to specify a sub-portion of a SPECT Patient Positioning Monitor (PPM) image in which to acquire CT data.

15. The method of claim 14, further including limiting the extent of the scan of the second imaging device based on said sub-portion so as to limit dose delivered to the patient by the second imaging device.

16. The method of claim 6, further including acquiring a Topogram with the second imaging device and using said Topogram in conjunction with SPECT Patient Positioning Monitor (PPM) information for patient positioning with respect to the second imaging device.

17. The method of claim 16, wherein said using said Topogram in conjunction with SPECT PPM information includes displaying PPM information along with the Topogram image.

18. The method of claim 17, further including displaying an extent of the PPM Field of View as an annotation overlaid on the Topogram.

19. The method of claim 17, further including storing a PPM image and overlaying said PPM image with said Topogram.

20. The method of claim 6, further including enabling the user to set the extent of a scan of the second imaging device.

21. The method of claim 6, further including said system treating a Field of View determined by SPECT Patient Positioning Monitor (PPM) images as initial settings for an extent of a scan of the second imaging device.

22. The method of claim 21, further including said system enabling a user to modify the extent to refine the area to perform the scan.

23. The method of claim 22, further including displaying an original Patient Positioning Monitor (PPM) Field of View even when the user has modified the extent to enable a user to keep track thereof.

24. A computer readable medium having a set of instructions for performing patient positioning in a hybrid medical imaging system comprising:
a first set of instructions for concurrently presenting on a display images from both a first nuclear medical imaging device and a second imaging device to aid in patient positioning with respect to both the nuclear medical imaging device and the second imaging device;
a second set of instructions for positioning a Field of View of the nuclear medical imaging device; and
a third set of instructions for adapting a scan of the second medical imaging device with respect to the Field of View of the nuclear medical imaging device.

25. A computer readable medium of claim 24, wherein the third set of instructions further comprises:
a fourth set of instructions for adapting the scan of the second medical imaging device to match the Field of View of the nuclear medical imaging device.

26. A computer readable medium of claim 24, wherein the third set of instructions further comprises:
a fifth set of instructions for adapting the scan of the second medical imaging device to a sub-portion of the Field of View of the nuclear medical imaging device.
